(19) United States
(12) Patent Application Publication
(10) Pub. No.: US 2006/0276390 A1
(43) Pub. Date: Dec. 7, 2006

(54) Combined treatments comprising synthetic peptide copolymers for preventing graft rejection

(75) Inventors: Rina Aharoni, Rehovot (IL); Ruth Arnon, Rehovot (IL); Michael Sela, Rehovot (IL); Alex Yussim, Tel Aviv (IL)

Correspondence Address: HESLIN ROTHENBERG FARLEY & MESITI PC, 5 COLUMBIA CIRCLE, ALBANY, NY, 12203, US

(73) Assignees: YEDA RESEARCH AND DEVELOPMENT CO. LTD., REHOVOT (IL); MOR RESEARCH APPLICATIONS LTD., PETACH TIKVA (IL)

(21) Appl. No.: 10/566,321

(22) PCT Filed:
(86) PCT No.: PCT/IL04/00695

Related U.S. Application Data
(60) Provisional application No. 60/491,236, filed on Jul. 31, 2003.

(51) Int. Cl. A61K 38/16; A61K 38/13; A61K 39/395; A61K 31/522; A61K 31/4745

(52) U.S. Cl. 514/12; 514/11; 514/263.31; 514/291; 424/131.1

(57) ABSTRACT

Compositions and methods for the treatment of graft rejection associated with transplantation of tissues and organs include combined treatment involving at least one agent selected from Copolymer 1, a copolymer 1-related heteropolymer or an ordered peptide in combination with at least one additional known immunosuppressive agent. Compositions and methods for the treatment of graft rejection using ordered peptides or ordered copolymer 1-related heteropolymers as monotherapy are also provided.

### FIELD OF THE INVENTION

The present invention provides compositions and methods for the prevention and treatment of graft rejection, and for attenuating host responses in transplantation of tissues and organs. More specifically, the compositions and methods of the present invention relate to combined modalities of treatment involving at least one heteropolymer of amino acids or one ordered peptide in conjunction with at least one additional known immunosuppressive agent.

### BACKGROUND OF THE INVENTION

Transplantation systems such as organ transplantations and bone marrow reconstitution have become important and effective therapies for many life-threatening diseases. However, immune rejection is still the major barrier for successful transplantation. This is manifested in functional deterioration and graft rejection in the case of organ transplantation (host-versus-graft response, or HVG). Another manifestation of pathological immune reactivity is graft-versus-host disease (GVHD) that occurs in approximately 30% of bone marrow recipients. Thus, there is an unmet medical need in controlling or preventing HVG responses and GVHD.

Current available approaches for prevention of graft rejection include the use of non-specific immunosuppressive drugs, such as cyclosporins including cyclosporin A (CyA), tacrolimus (also known as FK506), methotrexate and/or prednisone. However, these treatments induce severe side effects, including nephrotoxicity, hypertension, hypercholesterolemia, diabetogenic effects, neurotoxicity, hirsutism and gingival hyperplasia. Moreover, the non-selective depression of the entire immune system renders patients vulnerable to infections. Despite chronic administration of immunosuppressive agents, transplantations have achieved only limited success as a therapeutic approach for long term survival. Given these limitations, traditional immunosuppressive therapies cannot overcome the more aggressive rejection process of HLA unmatched transplants and xenografts. Hence, these traditional therapies do not solve the problem of the acute and growing shortage of human donors.

The pathological process of immune rejection is mediated by T cells that recognize alloantigens presented on self major histocompatibility complex (MHC) molecules, as non-self. They then proliferate, secrete cytokines, and recruit additional inflammatory and cytotoxic cells (Sykes et al., 1996). In order to prevent immune rejection, it is therefore essential to inhibit antigen presentation and consequently T cell activation. It has been demonstrated that small synthetic peptides of 11-14 amino acids with high binding affinity for specific class II MHC molecules, were capable of preventing murine graft-versus-host disease (Schlegel et al., 1994). This approach, however, has been limited by the need for allelic specificity of the inhibitor peptides to the MHC haplotype of the donor/recipient, as well as by the difficulty of achieving sustained tissue levels of such low molecular weight peptides over a prolonged period of time.

A high molecular weight synthetic basic random copolymer consisting of L-Ala, L-Glu, L-Lys and L-Tyr residues in the molar ratio of about 6 parts Ala to 2 parts Glu to 4.5 parts Lys to 1 part Tyr, and having a molecular weight of 15,000-25,000, was first described in U.S. Pat. No. 3,849,550 as an agent for treatment or prevention of experimental allergic encephalomyelitis (EAE), a disease resembling multiple sclerosis (MS) that can be induced in susceptible animals.

D-Copolymer 1 or D-Cop 1, in which the four amino acids have the D-configuration, namely a random copolymer containing the D-Ala, D-Glu, D-Lys and D-Tyr residues, has also been described (Webb et al., 1976).

Copolymer 1 (Cop 1 also known by the trivial chemical name glatiramer acetate), a non-pathogenic synthetic random copolymer composed of the four amino acids: L-Glu, L-Lys, L-Ala, and L-Tyr (hereinafter “Cop 1”), is currently an approved drug for the treatment of multiple sclerosis under the name of COPAXONE® (Teitelbaum et al., 1998). It is very well tolerated with only minor adverse reactions. Treatment with Cop 1 by ingestion or inhalation is disclosed in U.S. Pat. No. 6,214,791.

Recently it was found that in animal models, Cop 1 provides a beneficial effect for several additional disorders. Thus, Cop 1 suppresses the immune rejection manifested in graft versus host disease (GVHD) in case of bone marrow transplantation (Schlegel et al., 1996; U.S. Pat. No. 5,858,964), as well as in graft rejection in case of solid organ transplantation (Aharoni et al., 2001; WO 00/27417).

WO 01/52878 and WO 01/93893 disclose that Cop 1, Cop 1-related peptides and polypeptides and T cells activated therewith protect CNS cells from glutamate toxicity and prevent or inhibit neuronal degeneration or promote nerve regeneration in the central nervous system (CNS) and peripheral nervous system (PNS). Thus, for example, Cop 1 is under evaluation as a therapeutic vaccine for neurodegenerative diseases such as optic neuropathies and glaucoma (Kipnis and Schwartz, 2002).

Cop 1 and related copolymers and peptides have been disclosed in WO 00/05250 (Aharoni et al., 2000), hereby incorporated by reference in its entirety as if fully disclosed herein, for treating autoimmune diseases.

WO 00/27417 discloses compositions and methods for treating and preventing host-versus-graft immune responses and graft-versus-host diseases comprising as an active ingredient Copolymer 1 and Copolymer 1-related random heteropolymers.

There exists a long-felt need for safer and more effective means of preventing or treating graft rejection. The present invention satisfies this need and provides related advantages as well.

### SUMMARY OF THE INVENTION

The present invention provides pharmaceutical compositions for use in the prevention and treatment of graft rejection (also referred to herein as host-versus-graft responses, abbreviated HVG). The compositions of the present invention comprise random or ordered copolymers including Copolymer 1 and Copolymer 1-related heteropolymers or ordered peptides, in combination with at least one additional known immunosuppressive agent.

The present invention is based in part on the surprising discovery that Copolymer 1 or Copolymer 1-related heteropolymers in combination with at least one additional immunosuppressive drug exhibit an unexpected synergistic effect for the treatment or prevention of HVG. According to the present invention, Copolymer 1 as well as Copolymer 1-related heteropolymers or peptides in combination with other immunosuppressive drugs induce an unexpected synergistic effect, and thus improve the efficacy of the current immunosuppressive regimens. Thus, the use of Copolymer 1, Copolymer 1-related heteropolymers in combination with other immunosuppressive drugs increases the effectiveness of the immunosuppressive drugs at lower dosages, thereby decreasing the toxic side effects. The combination of drugs may be administered together or may be administered sequentially. It is to be explicitly understood that present invention explicitly encompasses co-administration of these agents in a substantially simultaneous manner, as in a single unit dosage form suitable for oral or parenteral administration having a fixed ratio of these active agents or in multiple, separate unit dosage forms for each agent, each of which may independently be in a form suitable for oral administration or parenteral injection.

As disclosed herein, one aspect of the present invention provides methods of using Copolymer 1 or Copolymer 1-related heteropolymers in combination with additional immunosuppressive drugs for the treatment or prevention of graft rejection. According to the present invention, Copolymer 1 or Copolymer 1-related heteropolymers in combination with other immunosuppressive drugs induce a synergistic effect and thus enable the reduction in the dosage and toxicity of the current immunosuppressive regimens. The immunosuppressive drugs that are currently used for human transplantation induce severe and toxic side effects, which limit their application. Furthermore, whereas Cop 1 activity involves MHC blocking as well as Th1 to Th2 cytokine shift, general immunosuppressive drugs, such as cyclosporin A, tacrolimus (FK 506) and rapamycin interfere with signal transduction pathways. Without wishing to be bound by any particular theory or mechanism of action, Cop 1 in combination therapy with other immunosuppressive drugs may therefore improve the efficacy of the current immunosuppressive regimens.

It is now disclosed for the first time that surprisingly the beneficial effects of treatment of Cop1 in combination with additional immunosuppressive drugs is virtually identical to that obtained with pretreatment using classical immunosuppressive drugs prior to organ or cell transplantation, therefore obviating the need for such pretreatment and thereby reducing the side effects that might occur during pretreatment.

According to various embodiments, several groups of immunosuppressive drugs may be used in combination with Copolymer 1 or Copolymer 1-related heteropolymers according to the present invention. In one embodiment, drugs which are inhibitors of lymphocyte activation are used in the combination therapy. Preferred drugs are for example cyclosporin, preferably cyclosporin A, tacrolimus (FK 506), ISA247 or FK 778. In another embodiment, antiproliferative drugs are used in the combination therapy. Preferred drugs are for example rapamycin and everolimus (Certican®). In yet another embodiment, immunomodulators such as FTY720 which modulates lymphocyte re-circulation are used in the combination therapy. Other drugs, such as steroids, purine antimetabolites and antibodies may also be used in the combination therapy.

According to one embodiment of the present invention, the glatiramer acetate and Copolymer 1-related heteropolymers to be used in combination with additional immunosuppressive drugs comprise copolymers having random amino acid sequence (random copolymers).

According to another embodiment of the present invention, the agent to be used in combination with additional immunosuppressive drugs comprises peptides having ordered amino acid sequence (ordered peptides or ordered copolymers).

In another embodiment, the ordered peptides may be used as a mono-therapy for treating HVG. This embodiment of the present invention is based on the principle that specific ordered peptides, that may be considered Copolymer-1 related peptides, can be used as the active ingredient for monotherapy of HVG. Specifically, the inventors of the present application disclose herein for the first time the use of ordered peptides and ordered Copolymer 1-related heteropolymers for the treatment of HVG.

According to various embodiments of the present invention, the random or ordered copolymers and peptides to be used in the combination therapy comprise a suitable quantity of an amino acid of positive electrical charge, such as lysine or arginine, in combination with an amino acid with a negative electrical charge (preferably in a lesser quantity), such as glutamic acid or aspartic acid, optionally in combination with an electrically neutral amino acid such as alanine, glycine or valine, serving as a filler, and optionally with phenylalanine, tyrosine or tryptophan, the optional amino acids adapted to confer on the copolymer immunogenic properties.

The copolymers to be used in the combination therapy can be composed of L- or D-amino acids or mixtures thereof. As is known by those of skill in the art, L-amino acids occur in most natural proteins. However, D-amino acids are commercially available and can be substituted for some or all of the amino acids used to make the copolymers used in the present invention. The present invention contemplates the use of copolymers containing both D- and L-amino acids, as well as copolymers consisting essentially of either L- or D-amino acids.

In various embodiments of the present invention, the copolymer may be a random polypeptide from about 15 to about 100 amino acids, preferably from about 40 to about 80 amino acids in length In alternative embodiments, the agent is an ordered synthetic peptide of from 6 to 25 amino acids, preferably from 10 to 20 amino acids. In yet other embodiments oligomeric forms of these the peptides may be produced having from about 15 to about 100 amino acids, preferably from about 40 to about 80 amino acids in length.

More specifically, in one embodiment of the invention, the pharmaceutical composition to be used in the combination therapy for preventing and treating HVG comprises at least one random or ordered copolymer, said copolymer comprising at least three different amino acids, each selected from a different one of the following groups:

(a) lysine and arginine;

(b) glutamic acid and aspartic acid;

(c) alanine, glycine and valine;

(d) phenylalanine, tyrosine and tryptophan,

A preferred copolymer for use in the combination therapy comprises in combination alanine, glutamic acid, lysine, and tyrosine, of net overall positive electrical charge. In a preferred embodiment, the pharmaceutical composition comprises Copolymer 1 of molar ratio of the amino acids glutamic acid about 0.14, alanine about 0.43, tyrosine about 0.10, and lysine about 0.34. In another preferred embodiment, preferred molar ratios of the amino acid residues include the relative molar ratios 0.17 glutamic acid to 0.38 lysine to 0.49 alanine to 0.1 tyrosine, or 0.19 glutamic acid to 0.4 lysine to 0.6 alanine to 0.1 tyrosine.

In one embodiment, average molecular weight of the copolymer of the invention is about 2,000-40,000 Da, preferably of about 2,000-18,000 Da, more preferably of about 4,500-16,000 Da. According to some embodiments glatiramer acetate used in the compositions or methods of the invention more preferably has an average molecular weight of about 5,000-9,000 Da, and most preferred of about 6,000-8,000 Da.

It is clear that this is given by way of example only, and that the composition can be varied both with respect to the constituents and relative proportions of the constituents if the above general criteria are adhered to.

In another embodiment, the copolymer for use in the combination therapy contains three different amino acids each one selected from three groups of the groups (a) to (d). These copolymers are herein referred to as terpolymers.

Thus, the present invention is also directed to pharmaceutical compositions for use in the combination therapy comprising a therapeutically effective amount of at least one random or ordered terpolymer. The terpolymer consists of three different amino acids, each selected from a different one of the following groups:

(a) lysine and arginine;

(b) alanine, glycine and valine;

(c) phenylalanine, tyrosine or tryptophan.

A preferred copolymer according to this embodiment of the present invention contains tyrosine, alanine and lysine, in the molar ratio of from about 0.005 to about 0.25 tyrosine, from about 0.3 to about 0.6 alanine, and from about 0.1 to about 0.5 lysine, along with a pharmaceutically acceptable carrier. This terpolymer, hereinafter designated YAK, is preferably substantially free of glutamic acid.

In a preferred embodiment, the molar ratios of tyrosine, alanine and lysine are about 0.10 to about 0.54 to about 0.35, respectively. The average molecular weight of YAK is about 2,000-40,000 Da, preferably about 3,000-35,000 Da, more preferably about 5,000-25,000 Da. It is possible to substitute arginine for lysine, glycine or valine for alanine or phenylalanine or tryptophan for tyrosine.

The present invention further provides a pharmaceutical composition which includes a therapeutically effective amount of a random or ordered terpolymer for use in the combination therapy consisting of three different amino acids, each selected from a different one of the following groups:

(a) lysine and arginine;

(b) glutamic acid and aspartic acid;

(c) phenylalanine, tyrosine and tryptophan.

A preferred copolymer according to this embodiment of the present invention contains glutamic acid, tyrosine, and lysine, in the molar ratio of from about 0.005 to about 0.300 glutamic acid, from about 0.005 to about 0.250 tyrosine, and from about 0.3 to about 0.7 lysine, and a pharmaceutically acceptable carrier. This terpolymer, hereinafter designated YEK, is preferably substantially free of alanine.

In a preferred embodiment, the molar ratios of glutamic acid, tyrosine, and lysine are about 0.26 to about 0.16 to about 0.58, respectively. The average molecular weight of YEK is about 2,000-40,000 Da, preferably about 3,000-35,000 Da, more preferably about 5,000-25,000 Da. It is possible to substitute arginine for lysine, aspartic acid for glutamic acid or phenylalanine or tryptophan for tyrosine.

The present invention is also directed to pharmaceutical composition which include a therapeutically effective amount of a random or ordered terpolymer for use in the combination therapy consisting of three different amino acids, each selected from a different member of the following groups:

(a) glutamic acid and aspartic acid;

(b) alanine, glycine and valine;

(c) phenylalanine, tyrosine and tryptophan.

A preferred copolymer according to this embodiment of the present invention contains tyrosine, glutamic acid and alanine, in the molar ratio of from about 0.005 to about 0.25 tyrosine, from about 0.005 to about 0.3 glutamic acid, and from about 0.005 to about 0.8 alanine, and a pharmaceutically acceptable carrier. This terpolymer, hereinafter designated YEA, is preferably substantially free of lysine.

In a preferred embodiment, the molar ratios of glutamic acid, alanine, and tyrosine are about 0.21 to about 0.65 to about 0.14, respectively. The average molecular weight of YEA is about 2,000-40,000 Da, preferably about 3,000-35,000 Da, and more preferably about 5,000-25,000 Da It is possible to substitute aspartic acid for glutamic acid, glycine for alanine, and phenylalanine or tryptophan for tyrosine.

The present invention further provides methods for treating and preventing HVG in a mammal by administering a therapeutically effective amount of a composition comprising at least one copolymer as described above in combination with at least one additional immunosuppressive drug, said copolymer selected from the group consisting of random, copolymers are ordered copolymers, said copolymer comprising at least three different amino acids each selected from at least three of the following groups:

(a) lysine and arginine;

(b) glutamic acid and aspartic acid;

(c) alanine, glycine and valine;

(d) phenylalanine, tyrosine and tryptophan.

Furthermore, the present invention is based on the surprising discovery that specific ordered Copolymer 1-related heteropolymers can be used as a single active ingredient for the treatment of HVG. Specifically, the inventors of the present application disclose herein for the first time the use of ordered Copolymer 1-related heteropolymers for the treatment of HVG.

As indicated hereinabove, heteropolymers having ordered amino acid sequence (ordered copolymers) are within the scope of the present invention. Examples of such heteropolymers or peptides are those disclosed in WO 00/05249, the entire contents of which being hereby incorporated herein by reference. Thirty-two of the peptides specifically disclosed in said application are reproduced in Table 1, hereinbelow. Such peptides and other similar peptides are expected to have similar activity as Cop 1. Such peptides, and other similar peptides, are also considered to be within the definition of Cop 1-related peptides or polypeptides and their use is considered to be part of the present invention.

TABLE 1

ORDERED PEPTIDES

| | |
|---|---|
| SEQ ID NO. | Peptide Sequence |

| | |
|---|---|
| 1 | AAAYAAAAAAKAAAA |
| 2 | AEKYAAAAAAKAAAA |
| 3 | AKEYAAAAAAKAAAA |
| 4 | AKKYAAAAAAKAAAA |
| 5 | AEAYAAAAAAKAAAA |
| 6 | KEAYAAAAAAKAAAA |
| 7 | AEEYAAAAAAKAAAA |
| 8 | AAEYAAAAAAKAAAA |
| 9 | EKAYAAAAAAKAAAA |
| 10 | AAKYEAAAAAKAAAA |
| 11 | AAKYAEAAAAKAAAA |
| 12 | EAAYAAAAAAKAAAA |
| 13 | EKKYAAAAAAKAAAA |
| 14 | EAKYAAAAAAKAAAA |
| 15 | AEKYAAAAAAAAAAA |
| 16 | AKEYAAAAAAAAAAA |
| 17 | AKKYEAAAAAAAAAA |
| 18 | AKKYAEAAAAAAAAA |
| 19 | AEAYKAAAAAAAAAA |
| 20 | KEAYAAAAAAAAAAA |
| 21 | AEEYKAAAAAAAAAA |
| 22 | AAEYKAAAAAAAAAA |
| 23 | EKAYAAAAAAAAAAA |
| 24 | AAKYEAAAAAAAAAA |
| 25 | AAKYAEAAAAAAAAA |
| 26 | EKKYAAAAAAAAAAA |
| 27 | EAKYAAAAAAAAAAA |
| 28 | AEYAKAAAAAAAAAA |
| 29 | AEKAYAAAAAAAAAA |
| 30 | EKYAAAAAAAAAAAA |
| 31 | AYKAEAAAAAAAAAA |
| 32 | AKYAEAAAAAAAAAA |

In various embodiments of the present invention, the prevention and/or treatment of host-versus-graft rejection includes transplantation of organs or tissues from BLA matched or unmatched allogeneic human donors, or xenografts from donors of other species.
In one embodiment, host-versus-graft rejection includes the rejection of transplanted cells, tissue or organs selected from hematopoietic cells, stem cells, heart, lung, kidney, liver, skin and other organs or tissues transplanted from donor to recipient.
According to various embodiments of the present invention, the therapeutically effective amount of Copolymer 1-related heteropolymers are from about 1.0 mg to about 500.0 mg/day. Preferably, such therapeutically effective amounts of Copolymer 1-related heteropolymers are from about 20.0 mg to about 100.0 mg/day.

Although the present specification describes some preferred embodiments of the invention, it is to be understood that the present invention encompasses the use of any synthetic random or ordered copolymer of at least three of Glu or Asp, Lys or Arg, Ala Gly or valine, and Phe or Tyr or Trp in combination with an immunosuppressive agent, having a relative molar ration of the amino acid residues and an average molecular weight as defined herein, including those forms of Cop 1 described in the literature that fall within the definition of the present invention.

In another aspect, the invention relates to the use of the random or ordered copolymers described above in combination with an immunosuppressive agent for the manufacture of a medicament for prevention and treatment of graft rejection.

In a further embodiment, the invention relates to a method of treatment of HVG in the course of organ transplantation, said method comprises administering to a patient in need an effective amounts of the above-mentioned random or ordered copolymers in combination with at least one immunosuppressive agent.

According to various embodiments, several groups of immunosuppressive drugs may be used in combination with Copolymer 1 or Copolymer 1-related heteropolymers according to the present invention. In one embodiment, drugs which are inhibitors of lymphocyte activation are used in the combination therapy. Preferred drugs are for example cyclosporins, preferably cyclosporin A, tacrolimus, ISA247 or FK 778. In another embodiment, antiproliferative drugs are used in the combination therapy. Preferred drugs are for example rapamycin and everolimus. In yet another embodiment, immunomodulators such as FTY720 which modulate lymphocyte re-circulation are used in the combination therapy.

These and further embodiments will be apparent from the detailed description and examples that follow.

### BRIEF DESCRIPTION OF THE FIGURES

FIG. 1 depicts the effect of Cop 1 treatment on skin graft rejection in BALB/c mice receiving skin grafts from B10D2 donor mice. The percent survival of the B10D2 skin was used as a measure of skin graft rejection or acceptance. Cop 1 treatment was compared to treatment with PBS, and treatment with two known immunosuppressive agents, cyclosporin A (CyA) and tacrolimus (FK 506). The BALB/c recipient mice were treated daily with: (1) PBS injected i.p. (squares) from the seventh day prior to skin grafting; (2) Cop 1 injected daily i.p. and subcutaneously (sc) from the seventh day prior to skin grafting at a dosage of 300 micrograms/day (small circles) and 600 micrograms/day (large circles); (3) CyA injected i.p. (white triangles) at a dosage of 1 microgram/day from the seventh day prior to skin grafting and (4) tacrolimus injected seven times ip (black triangles) at a dosage of 300 micrograms/day from the second day prior to transplantation. Skin grafts were inspected daily. Rejection was considered positive when no viable donor epidermis remained.

FIG. 2 shows the effect of Cop 1 treatment on grafted thyroid function in BALB/c mice in which thyroid glands from B10D2 donor mice were transplanted into the kidney capsules. Cop 1 treatment (600 micrograms/day Cop 1 injected ip daily from 7^{th} day prior to transplantation) was compared to treatment with PBS (injected ip daily from 7^{th} day prior to transplantation), cyclosporin A (1 microgram/day Cy A injected ip daily from 7^{th} day prior to transplantation) and tacrolimus (300 micrograms/day injected ip seven times from 2^{nd} day prior to transplantation). One week from transplantation, the transplanted mice were injected with ¹²⁵I and the radioactivity of each was measured twenty hours later. The mean ¹²⁵I absorbance of the recipient kidneys (solid bars) and the mean ¹²⁵I absorbance of the untransplanted kidneys (striped bars) is depicted.

FIG. 3 demonstrates the effects of GA, CyA and tacrolimus on the survival of strongly mismatched skin grafts. BALB/c mice were transplanted with skin grafts from C57BL/6 donors. (A) The effect of GA in comparison to various doses of immunosuppressive drugs: GA 100 mg/kg starting 2 weeks before transplantation; CyA 5, 10 or 15 mg/kg, starting 6 days before transplantation; and tacrolimus 5 or 10 mg/kg, starting 6 days before transplantation. (B) The effect of GA in combination with immunosuppressive drugs: GA 100 mg/kg was administered with either CyA 7.5 mg/kg or tacrolimus 5 mg/kg. The effect of the combined treatment in comparison to the immunosuppressive drugs alone is demonstrated. Grafts were considered rejected when no viable donor epidermis remained. At least 7 mice were tested in each group. Statistical significance for graft survival over untreated control (p<0.05 by Kaplan-Meier test) was obtained for GA, CyA 15 mg/kg and tacrolimus 10 mg/kg (A), and for the two combination treatments (B). (C) Depicts one mouse treated by a combination of GA and FK 506 in which engraftment was sustained for 45 days even though treatment was discontinued 20 days after transplantation.

FIG. 4 shows the effect of Cop 1 and combination treatments with CyA (a) and FK 506 (b), on the function of transplanted thyroids from B10D2 donor mice, which were grafted into the kidney capsules of BALB/c mice. The mean ¹²⁵I absorbance of the recipient kidneys (solid bars) and the mean ¹²⁵I absorbance of the untransplanted kidneys striped bars) is depicted. The numbers represent the mean functional index (MFI described in materials and methods)

FIG. 5 demonstrates the effects of GA, CyA and tacrolimus (FK 506) on Lewis rats that were transplanted with an accessory heart from allogeneic disparate BN donor rats. Recipient rats were treated daily with one of the following treatment regimens: GA 100 mg/kg starting 2 weeks before transplantation; CyA 1.25, 2.5, 5 or 10 mg/kg starting on the day of transplantation (shaded bars, FIG. 5A); or tacrolimus 1.25, 2.5 or 5 mg/kg starting 6 days before transplantation (shaded bars, FIG. 5B); Combination of pretreatment with GA and the respective doses of the immunosuppressants (open bars, see FIGS. 5A and 5B); Treatment with GA and administered from the day of transplantation without pretreatment (striped bar, FIG. 5B). Cardiac allograft survival was inspected daily by monitoring palpation of the grafts. Grafts were considered rejected when no heart palpitations could be noticed. Groups of 5-15 rats were used for each point.

### DETAILED DESCRIPTION OF THE INVENTION

The nomenclature GLAT copolymer or YEAK copolymer has also been used for Cop 1 known by the trivial chemical name glatiramer acetate (GA). Thus, hereinafter in the specification and in the claims, the terms Copolymer 1, Cop 1, L-GLAT and L-YEAK will be used interchangeably for the L form of Cop 1, and the terms D-Copolymer 1, D-Cop 1, D-GLAT and D-YEAK will be used interchangeably for the D form of Cop 1.

The phrase “combination therapy” in defining the use of immunosuppressive drugs in combination with Copolymer 1 or Copolymer 1-related heteropolymers, is intended to embrace administration of each agent in a sequential manner in a regimen that will provide beneficial effects of the drug combination. The phrase also is intended to embrace co-administration of these agents in a substantially simultaneous manner, such as in a single capsule having a fixed ratio of these active agents or in multiple, separate capsules for each agent.

The phrase “therapeutically effective amounts” is intended to qualify the amount of each agent for use in the combination therapy which will achieve the goal of improvement in severity and the frequency of incidence over treatment of each agent by itself, while avoiding adverse side effects typically associated with alternative therapies.

The random and ordered Copolymer 1 and Copolymer 1-related copolymers used in the combination therapy of the present invention represent a novel therapeutic approach to treat graft rejection. Specifically, random and ordered copolymers are used in the combination therapy for the treatment of HVG, particularly with regard to cell, tissue and organ transplantation, selected from hematopoietic cells, stem cells, hearts, lungs, kidneys, livers, skin and other organs or tissues transplanted from donor to recipient.

The copolymers for use in the present invention can be composed of L- or D-amino acids or mixtures thereof. As is known by those of skill in the art, L-amino acids occur in most natural proteins. However, D-amino acids are commercially available and can be substituted for some or all of the amino acids used to make the terpolymers and other copolymers of the present invention. The present invention contemplates copolymers containing both D- and L-amino acids, as well as copolymers consisting essentially of either L- or D-amino acids.

The average molecular weight and the average molar fraction of the amino acids in the copolymers can vary. However, a molecular weight range of about 2,000 to 40,000 daltons is contemplated. A preferred molecular weight range is from about 2,000 to about 18,000 daltons. The copolymers can be from about 15 to about 100 amino acids, preferably from about 40 to about 80 amino acids in length. Preferred molecular weight ranges and processes for making a preferred form of Copolymer 1 is described in U.S. Pat. Nos. 5,800,808 and 5,858,964 the entire contents of which are hereby incorporated in the entirety.

In one embodiment, the terpolymers for use in the combination therapy of the present invention contain tyrosine, alanine, and lysine, hereinafter designated YAK. The average molar fraction of the amino aids in these terpolymers can vary. For example, tyrosine can be present in a mole fraction of about 0.005 to about 0.250; alanine can be present in a mole fraction of about 0.3 to about 0.6; and lysine can be present in a mole fraction of about 0.1 to about 0.5. The average molecular weight is between 2,000 to about 40,000 daltons, and preferably between about 3,000 to about 35,000 daltons. In a more preferred embodiment, the average molecular weight is about 5,000 to about 25,000 daltons. It is possible to substitute arginine for lysine, glycine for alanine or phenylalanine or tryptophan for tyrosine.

In another embodiment, the terpolymers for use in the combination therapy of the present invention contain tyrosine, glutamic acid, and lysine, hereinafter designated YEK. The average molar fraction of the amino acids in these terpolymers can vary: glutamic acid can be present in a mole fraction of about 0.005 to about 0.300, tyrosine can be present in a mole fraction of about 0.005 to about 0.250, lysine can be present in a mole fraction of about 0.3 to about 0.7. The average molecular weight is between 2,000 and about 40,000 daltons, and preferably between about 3,000 and about 35,000 daltons. In a more preferred embodiment, the average molecular weight is about 5,000 to about 25,000 daltons. It is possible to substitute aspartic acid for glutamic acid, arginine for lysine or phenylalanine or tryptophan for tyrosine.

In another embodiment, the terpolymers for use in the combination therapy of the present invention contain tyrosine, glutamic acid, and alanine, hereinafter designated YEA. The average molar fraction of the amino acids in these polypeptides can vary. For example, tyrosine can be present in a mole fraction of about 0.005 to about 0.250, glutamic acid can be present in a mole fraction of about 0.005 to about 0.300, and alanine can be present in a mole fraction of about 0.005 to about 0.800. The average molecular weight is between 2,000 and about 40,000 daltons, and preferably between about 3,000 and about 35,000 daltons. In a more preferred embodiment, the average molecular weight is about 5,000 to about 25,000 daltons. It is possible to substitute aspartic acid for glutamic acid and glycine for alanine.

In a more preferred embodiment, the mole fraction of amino acids of the heteropolymers for use in the combination therapy is about what is preferred for Copolymer 1. The mole fraction of amino acids in Copolymer 1 is glutamic acid about 0.14, alanine about 0.43, tyrosine about 0.10, and lysine about 0.34. The most preferred average molecular weight for Copolymer 1 is between about 5,000 and about 9,000 daltons. The activity of Copolymer 1 in the treatment of HVG is expected to remain if one or more of the following substitutions is made: aspartic acid for glutamic acid, glycine for alanine, and arginine for lysine

The molar ratios of the monomers of the more preferred terpolymer of glutamic acid, alanine, and tyrosine, or YEA, is about 0.21 to about 0.65 to about 0.14.

The molar ratios of the monomers of the more preferred terpolymer of glutamic acid, tyrosine, and lysine, or YEK, is about 0.26 to about 0.16 to about 0.58.

The molar ratios of the monomers of the more preferred terpolymer of tyrosine, alanine and lysine, or YAK, is about 0.10 to about 0.54 to about 0.35.

According to the present invention, the limitations of currently available immunosuppression therapies used in patients about to undergo organ transplantation are overcome by use of Copolymer 1 or other random or ordered copolymers as described herein. Copolymer 1 has been approved in several countries for the treatment of Multiple Sclerosis (MS) under the trade name, Copaxone®, Glatiramer acetate. Several clinical trials demonstrated that Copolymer 1 is well tolerated with only minor side reactions which were mostly mild reactions at the injection site (Johnson et al., 1995).

According to the present invention, L-Copolymer 1, D-Copolymer 1 and other random and ordered copolymers are envisaged to prevent or significantly delay graft rejection when administered in combination with immunosuppressive agents. As shown in the Examples hereinafter, Copolymer 1 is effective in suppressing in mice the rejection of grafts received from another mouse strain of the same MHC haplotype. Thus, graft rejection could be suppressed in BALB/c mice receiving grafts from B10.D2 donor mice, in C3HSH mice receiving grafts from C57BL donor mice, and in PJL mice receiving grafts from B10PL donor mice (Tables 4 and 5). These transplantation mouse models are similar to the MHC matched organ transplantation in humans. Moreover, Copolymer 1 is also effective in suppressing in mice rejection of grafts from strains of different MHC haplotypes, for example, suppressing in BALB/C mice rejection of grafts received from C57BL donor mice (see Tables 4 and 5 herein), a model which is similar to the MHC unmatched organ transplantation in humans. Thus, pre and post transplantation administration of Copolymer 1 over a limited time after transplantation can significantly reduce the incidence, onset and severity of immunorejection, resulting in improved long-termed survival.

As described before for the mechanism of action for GVHD (Aharoni et al., 1), Copolymer 1 inhibits T cell proliferation in response to graft cells. Copolymer 1 treatment completely abolished cytotoxic activity towards graft cells, preventing the secretion of cytokines like interleukin 2 (IL-2) and interferon γ (IFN-γ), and induced a beneficial anti-inflammatory response.

The present invention is also directed to the use of terpolymers as defined herein for the prevention and treatment of HVG when administered in combination with immunosuppressive agents. The terpolymers can be made by any procedure available to one of skill in the art. For example, the terpolymers can be made under condensation conditions using the desired molar ratio of amino acids in solution, or by solid phase synthetic procedures. Condensation conditions include the proper temperature, pH, and solvent conditions for condensing the carboxyl group of one amino acid with the amino group of another amino acid to form a peptide bond. Condensing agents, for example, dicyclohexyl-carbodiimide, can be used to facilitate the formation of the peptide bond. Blocking groups can be used to protect functional groups, such as the side chain moieties and some of the amino or carboxyl groups against undesired side reactions.

As disclosed hereinabove, one aspect of the present invention is the use of Copolymer 1 or Copolymer 1-related heteropolymers in combination with additional immunosuppressive drugs for the treatment of graft rejection. According to the present invention, Copolymer 1 or Copolymer 1-related heteropolymers in combination with other immunosuppressive drugs induce a synergistic effect and thus enable the reduction in the dosage and toxicity of the current immunosuppressive regimens. The immunosuppressive drugs that are currently used for human transplantation induce severe and toxic side effects, which limit their application. Furthermore, whereas Cop 1 activity involves MHC blocking as well as Th1 to Th2 cytokine shift, immunosuppressive drugs, such as cyclosporin A, tacrolimus (also known as FK 506) and rapamycin interfere with signal transduction pathways. Cop 1 in combination therapy with other immunosuppressive drugs may therefore induce an additive or synergistic effect, and thus improve the efficacy of the current immunosuppressive regimens.

According to the present invention, three experimental animal models were used, each one having its own particular features: namely skin grafting, thyroid transplantation and heterotopic heart transplantation: 1. Skin grafting is one of the most stubborn tissues in transplantation. The only parameter for success is mean survival time (MST) of the graft and even a modest prolongation is considered meaningful. 2. Thyroid transplantation allows the evaluation of functionality of the graft by its capacity to absorb iodine. The functionality is defined by the net extent of radioactive iodine absorption in the transplanted kidney (after subtraction of the radioactivity in the non-transplanted one and hence a built-in control is provided in each individual mouse. The effect of the treatment by the various drugs and combinations in this system is expressed numerically as the mean function index (MFI), which is the ratio between the net iodine absorbance in treated versus untreated mice, but is thus based entirely on the biological functionality of the graft. 3. Heterotopic heart transplant in rats allows the study of vascularised and perfused organs in animals larger than mice. It was previously demonstrated that different rejection behaviors are obtained towards vascularized and unvascularized allografts, presumably due to the different routes of antigen presentation by the different graft (Morris P J, Wood K J, Dallman M J. Antigen-induced tolerance to organ allografts. Annals of the New York Academy of Science 1991; 636: 295). This animal model for heart transplantation, which enables monitoring the survival and activity of the transplanted grafts by assessing heart palpation, is therefore more relevant to organ transplantation in patients.

According to the present invention, in all three transplantation systems (skin-graft and thyroid transplantation in mice, as well as heart transplantation in rats) combination therapy with Cop1, with either CyA or tacrolimus, was significantly effective. The delay obtained in graft rejection was in all cases higher than that obtained with Cop1 alone or with the immunosuppressive drugs alone. Moreover, in all cases, the combination with Cop1 was more effective than at least a double dose of the immunosuppressive therapy alone. Most importantly, Cop1 in combination treatment with tacrolimus on heterotopic heart rejection on the day of transplantation was similarly effective compared to the results obtained with pretreatment using classical immunosuppressive drugs prior to organ or cell transplantation.

According to various embodiments, several groups of immunosuppressive drugs may be used in combination with Copolymer 1 or Copolymer 1-related heteropolymers according to the present invention. In one embodiment, drugs which are inhibitors of lymphocyte activation are used in the combination therapy. Preferred drugs are for example cyclosporin, preferably cyclosporin A, tacrolimus (FK 506), ISA247 or FK 778. The dose of Cyclosporin A to be administered in the combination therapy may be from 0.1 mg/kg body weight/day to 1 g/kg body weight/day, preferably from 1 mg/kg body weight/day to 100 mg/kg body weight/day, more preferably 6-10 mg/kg body weight/day. The dose of FK 506 to be administered in the combination therapy may be from 0.001 mg/kg body weight/day to 10 mg/kg body weight/day, preferably from 0.01 mg/kg body weight/day to 1 mg/kg body weight/day, more preferably 0.1-0.15 mg/kg body weight/day.

In another embodiment, antiproliferative drugs are used in the combination therapy. Preferred drugs are for example rapamycin and everolimus (Certican®). The dose of rapamycin to be administered in the combination therapy may be from 0.02 mg/day to 200 mg/day, preferably from 0.2 mg/day to 20 mg/day, more preferably 2-6 mg/day.

In yet another embodiment, immunomodulators such as FTY720 which modulates lymphocyte re-circulation are used in the combination therapy. Other drugs, such as steroids, purine antimetabolites and antibodies may also be used in the combination therapy.

The process disclosed in U.S. Pat. No. 3,849,550, can be used for preparing the copolymers of the invention. For example, the N-carboxyanhydrides of tyrosine, alanine, γ-benzyl glutamate and N, ε-trifluoroacetyl-lysine are polymerized at ambient temperatures in anhydrous dioxane with diethylamine as an initiator. The γ-carboxyl group of the glutamic acid can be deblocked by hydrogen bromide in glacial acetic acid. The trifluoroacetyl groups are removed from lysine by one molar piperidine. One of skill in the art readily understands that the process can be adjusted to make peptides and polypeptides containing the desired amino acids, that is, three of the four amino acids in Copolymer 1, by selectively eliminating the reactions that relate to any one of glutamic acid, alanine, tyrosine, or lysine. U.S. Pat. Nos. 6,620,847; 6,362,161; 6,342,476; 6,054,430; 6,048,898 and 5,981,589 disclose improved methods for preparing glatiramer acetate (Cop-1). For purposes of this application, the terms “ambient temperature” and “room temperature” typically means a temperature ranging from about 20° C. to about 26° C.

The molecular weight of the terpolymers can be adjusted during polypeptide synthesis or after the terpolymers have been made. To adjust the molecular weight during polypeptide synthesis, the synthetic conditions or the amounts of amino acids are adjusted so that synthesis stops when the polypeptide reaches the approximate length which is desired. After synthesis, polypeptides with the desired molecular weight can be obtained by any available size selection procedure, such as chromatography of the polypeptides on a molecular weight sizing column or gel, and collection of the molecular weight ranges desired. The present polypeptides can also be partially hydrolyzed to remove high molecular weight species, for example, by acid or enzymatic hydrolysis, and then purified to remove the acid or enzymes.

In one embodiment, the terpolymers with a desired molecular weight may be prepared by a process which includes reacting a protected polypeptide with hydrobromic acid to form a trifluoroacetyl-polypeptide having the desired molecular weight profile. The reaction is performed for a time and at a temperature which is predetermined by one or more test reactions. During the test reaction, the time and temperature are varied and the molecular weight range of a given batch of test polypeptides is determined. The test conditions which provide the optimal molecular weight range for that batch of polypeptides are used for the batch. Thus, a trifluoroacetyl-polypeptide having the desired molecular weight profile can be produced by a process which includes reacting the protected polypeptide with hydrobromic acid for a time and at a temperature predetermined by test reaction. The trifluoroacetyl-polypeptide with the desired molecular weight profile is then further treated with an aqueous piperidine solution to form a low toxicity polypeptide having the desired molecular weight.

In a preferred embodiment, a test sample of protected polypeptide from a given batch is reacted with hydrobromic acid for about 10-50 hours at a temperature of about 20-28° C. The best conditions for that batch are determined by running several test reactions. For example, in one embodiment, the protected polypeptide is reacted with hydrobromic acid for about 17 hours at a temperature of about 26° C.

The random and ordered copolymers used in the present invention can be formulated into pharmaceutical compositions containing a pharmaceutically acceptable carrier. As used herein, “pharmaceutically acceptable carrier” includes any and all solvents, dispersion media, coatings, antibacterial and antifungal agents, isotonic and absorption delaying agents, sweeteners and the like. The pharmaceutically acceptable carriers may be prepared from a wide range of materials including, but not limited to diluents, binders and adhesives, lubricants, disintegrants, coloring agents, bulking agents, flavoring agents, sweetening agents and miscellaneous materials such as buffers and absorbents that may be needed in order to prepare a particular therapeutic composition. The use of such media and agents with pharmaceutically active substances well known in the art. Except insofar as any conventional media or agent is incompatible with the active ingredient, its use in the therapeutic compositions is contemplated.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example DMSO, or polyethylene glycol are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

Pharmaceutical compositions for parenteral administration include aqueous solutions of the active ingredients in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions.

U.S. Pat. No. 6,214,791 discloses methods for treating multiple sclerosis by oral administration of copolymer-1 through ingestion or inhalation. When copolymer-1 is introduced orally, it may be mixed with other food forms and consumed in solid, semi-solid, suspension, or emulsion form; and it may be mixed with pharmaceutically acceptable carriers, including water, suspending agents, emulsifying agents, flavor enhancers, and the like. In one embodiment, the oral composition is enterically-coated. Copolymer-1 may also be administered nasally in certain of the above-mentioned forms by inhalation or nose drops. Furthermore, oral inhalation may be employed to deliver copolymer-1 to the mucosal linings of the trachea and bronchial passages.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Materials and Methods

A. Preparation of Copolymer 1 and Controls

(i) Copolymer 1 was prepared by polymerization of the N-carboxyanhydrides of L-Ala, γ-benzyl-L-Glu, N,ε-trifluoroacetyl-L-Lys, and L-Tyr. The polymerization reaction was carried out at room temperature in anhydrous dioxane with diethylamine as initiator. Deblocking of the γ-carboxyl groups of the glutamic acid was carried out with hydrogen bromide in glacial acetic acid for 24 hours at room temperature, followed by removal of the trifluoroacetyl groups from the lysine residue by 1M piperidine. The end product is a mixture of acetate salts of random polypeptides with amino acid composition of Ala (4.1-5.8 residues), Glu (1.4-1.8 residues), Lys (3.2-4.2 residues), Tyr (1 residue).

(ii) The following peptides were synthesized by standard Fmoc chemistry. All peptides were 95% to 99% pure, as determined by high-performance liquid chromatography, and were checked by amino acid analysis and mass spectroscopy. Sequences are given in single letter codes:

MBP Ac1-11[4A], an acetylated N-terminal 1-11 peptide of myelin basic protein (MBP), with substitution of the original Lys residue at positio4 by Ala: AcASQARPSQRHG;

MBP 35-47, the epitope of MBP which is recognized in association with I-Eu: TGILDSIGRFFSG;

KM-core extension peptide, based on the antigenic core sequence of ovalbumin 323-339: KMKMVHAAHAKMKM;

MBP 89-101: VHFFKNIVTPRTP, was synthesized by t-butoxy-carbonyl chemistry.

B. Animals

BALB/c (H-2^{d}), B10.D2/nSnJ (H-2^{d}), CBA (H-2^{k}), C57BL/6 and C3H(H-2^{b}), B10.PL and PL/J (H-2^{μ}) mice were purchased from Jackson Laboratories (Bar Harbor, Me.); or from Simonsen Laboratories (Gilroy, Calif.).

C. Skin Grafts Transplantation Model System for HVG

Skin transplantation is an established model to measure immune rejection. In this model recipient and donor mice were anesthetized, shaved and cleaned. Circular pieces of skin (1.0 to 1.3 cm in diameter) were cut from dorsal side of the donor mice and dorsally transplanted to recipient animals by the use of histoacryl (Braun, Melsungen, Germany). The grafts were covered with Nobecutane antiseptic spray bandage (Astra, Wedel, Germany). Mice were kept in separate cages and inspected daily. Grafts were considered rejected when no viable donor epidermis remained.

D. Thyroid Graft Assay

In this transplantation model for HVG, thyroid glands from donor mice were transplanted in the kidney's capsules of recipient mice. One week later the transplanted mice were injected with ¹²⁵I, and the radioactivity of each kidney (the recipient or the untransplanted kidney) was measured after 20 hours. Δcpm was calculated by subtracting the ¹²⁵I absorbance of the untransplanted kidneys from the ¹²⁵I absorbance of the recipient kidneys in the same treatment. The mean function index (MFI) for treatment was calculated by dividing the mean Δcpm for the tested treatment by the mean Δcpm for the PBS treatment. P values were obtained by the ANOVA test. This assay indicates objectively and quantitatively not only the graft survival, but also the function (iodine absorbance) of the transplanted thyroid tissue (Isakov et al., 1979).

E. Copolymer 1 Treatment for HVG

The transplanted mice or rats were treated daily with Copolymer 1 at doses of 0.6-2.5 mg/day for mice and 25 mg/day for rats in PBS solution, starting 7 or 14 days before transplantation. In some cases, the first Cop 1 treatment was injected sc in ICFA as a depot dose.

F. Heterotopic Heart Transplantation

In this system rats (Lewis) were transplanted with additional heart from another strain (Fisher-344), using a cuff anastomosis technique. Cardiac allograft survival was monitored by daily palpation of the graft. Cessation of palpation indicated allograft rejection.

In all the transplantation systems the recipients were treated with daily doses of Cop 1 (COPAXONE® Teva, Israel) starting one or 2 weeks before transplantation, or immunosuppressive drugs i.e. Cyclosporin A (CyA, Sandoz Pharmaceuticals, East Hanover, N.J.) and tacrolimus (FK 506 Fujisawa) (Pharmaceuticals Osaka, Japan) starting 0-5 days before transplantation, in the indicated dosages.

### Example 1

### Effect of Copolymer 1 on Graft Rejection (HVG) in the B10.D2→BALB/c Model

The feasibility of using Copolymer 1 for the prevention of graft rejection was first tested on transplantation systems across minor histocompatibility barriers. Thus, recipient mice (BALB/C) were transplanted with grafts from another strain (B10.D2), but of the same H-2 haplotype (H-2^{d}), such that donors and recipients differed only in minor histocompatibility antigens (transplantation across minor histocompatibility barriers). This model closely resembles the clinical setting in the majority of human transplantations, in which donor and recipient are usually HLA matched.

The effect of Copolymer 1 was compared to the effect of control PBS treatment in two transplantation systems:

(i) Skin graft transplantation which usually results in a vigorous rejection process more difficult to suppress than other organ rejection (Isakov et al., 1979); and

(ii) Thyroid graft transplantation into the kidney's capsule which enables objective and quantitative induction not only of graft survival but also of the function (iodine uptake) of the transplanted thyroid tissue.

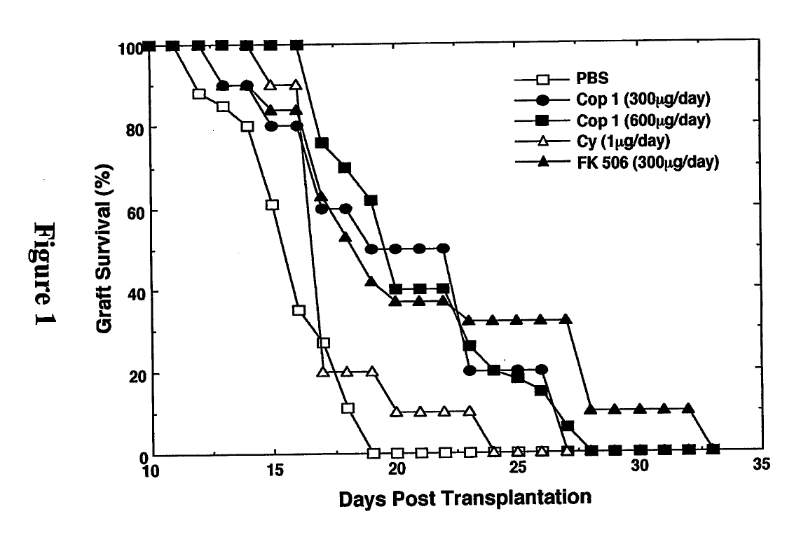

To test the effect of Copolymer 1 treatment on skin graft rejection in the B10.D2→BALB/c model, BALB/c recipient mice were transplanted with skin grafts from B10.D2 donors and were treated daily with: PBS ip from day −7, Copolymer 1 (ip+sc) from day −7. Grafts were inspected daily. Rejection was considered positive when no viable donor epidermis remained. The results are summarized in FIG. 1 and in Table 2. The mean graft survival time (MST) in Copolymer 1-treated mice was 20.4 days and 20.6 days, for 0.3 mg and 0.6 mg respectively, while the PBS control treatment resulted in MST of 16.1 days. Thus Cop 1 induced significant beneficial effect on skin graft survival in the B10D2→BALB/C system. It should be mentioned that Copolymer 1 was somewhat less effective than FK506 but more effective that CyA. However, a very low dose of CyA was used in this particular experiment (see FIG. 1).

To test the effect of Copolymer 1 treatment on the function of transplanted thyroids in the B10.D2→BALB/c model, thyroid glands from donors B10.D2 were transplanted in the kidney's capsules of BALB/c mice. After one week the transplanted mice were injected with I¹²⁵, and the radioactivity of each kidney was measured 20 hours later. A cpm was calculated by subtracting the I¹²⁵ absorbance of the untransplanted kidneys from the I¹²⁵ absorbance of the recipient kidneys in the same treatment. The mean function index (MFI) for each treatment was calculated by dividing the mean I¹²⁵ absorbance of (transplanted kidney—untransplanted kidney) in the tested treatment by the mean I¹²⁵ absorbance of (transplanted kidney—untransplanted kidney) in the PBS treatment, as follows:

$$* MFI = \frac{mean Δ cpm for the Cop 1 tested treatment /}{mean Δ cpm for the PBS tested treatment}$$

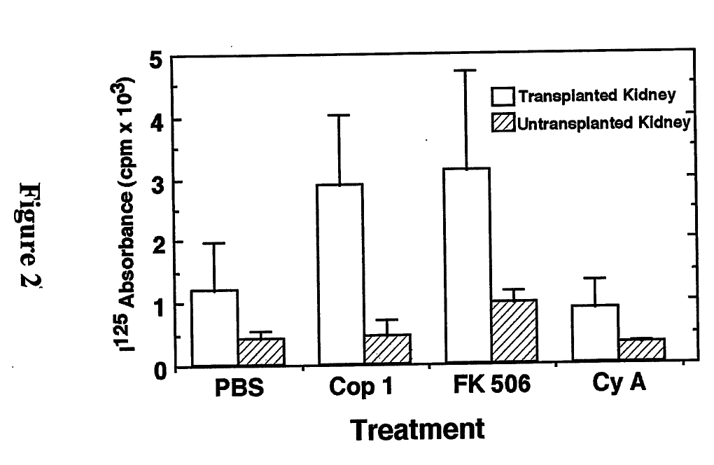

The results of thyroid transplantation are summarized in FIG. 2 and in Table 3. The MFI of the Copolymer 1-treated mice (600 ug/day) was 3.2 fold in one experiment and 5.2 fold in another experiment over PBS-treated mice. Thus Copolymer 1 treatment was significantly effective in preventing the functional deterioration of transplanted thyroid grafts in the B10D2→BALB/C system. This treatment was as effective as FK506 and much more effective than the low does of CyA used in this experiment.

These results show that Copolymer 1 induced significant and prominent effect on graft survival and function in both skin graft and thyroid transplantation systems.

Additional studies were then conducted with mice in order to establish the ability of Cop 1 to inhibit immune rejection of graft by host, using the two transplantation model systems described above while addressing the following aspects: (i) The effect of Copolymer 1 on HVG in different murine strain combinations; and (ii) The effect of Copolymer 1 on HVG in comparison to the effect of other immunosuppressive drugs that are currently used for human transplantation, namely FK506 and cyclosporin A.

TABLE 2
Effect of Copolymer 1 treatment on skin graft rejection in the B10.D2 →
BALB/c model

| Treatment | N | MST* ± SD | P** |
|---|---|---|---|
| PBS | 26 | 16.1 ± 2.2 | |
| Cop 1 300 μg/day | 10 | 20.4 ± 4.5 | <0.001 |
| Cop 1 600 μg/day | 34 | 20.6 ± 3.3 | <0.001 |
| Cy A 1 μg/day | 10 | 17.8 ± 2.4 | >0.05 |
| FK506 300 μg/day | 19 | 21.2 ± 4.3 | <0.001 |

*Mean Survival Time.
**P values were obtained by analysis of variance (ANOVA).

TABLE 3
Effect of Cop 1 treatment on the function of transplanted
thyroids in the B10.D2 → BALB/c model

| | | Mean I¹²⁵ Absorption | | | | |
|---|---|---|---|---|---|---|
| | | Transplanted | untransplanted | | | |
| Treatment | N | Kidney (cpm) | Kidney (cpm) | cpm | MFI* | P** |
| PBS | 10 | 1193 | 421 | 772 | 1.0 | |
| Cop 1 | 10 | 2901 | 450 | 2451 | 3.2 | 0.0005 |
| 600 μg | | | | | | |
| Cy A | 4 | 864 | 312 | 552 | 0.7 | 0.23 |
| 1 μg | | | | | | |
| FK 506 | 6 | 3117 | 693 | 2424 | 3.1 | 0.002 |
| 300 μg | | | | | | |

The mean function index (MFI) for each treatment was calculated as follows:

$* MFI = \frac{mean Δcpm for the tested treatment}{mean Δcpm for the PBS tested treatment}$
**P values were obtained by t test.

### Example 2

### The Effect of Copolymer 1 on HVG in Different Murine Strains

After testing the model of recipient/donor mice of different strains, but of the same H-2 haplotype, we tested rejection in mice transplanted with grafts from donors of another H-2 haplotype (transplantation across major histocompatibility barriers), a model of HLA unmatched transplantation in humans.

Thus, in order to find out whether the beneficial effect induced by Copolymer 1 on HVG represents a general phenomenon, we tested the ability of Copolymer 1 to inhibit graft rejection in additional strain combinations. Three murine strain combinations: B10.D2→BALB/c in the H-2^{d} haplotype, C57BL→C3HSW in the H-2^{b} haplotype, and B10PL→PL/J in the H-2^{u} haplotype, were tested across minor histocompatibility barriers, and across major histocompatibility barriers transplantation was tested in C57BL→BALB/c in the H-2^{b}→H-2^{d} haplotype. These strain combinations were tested with Copolymer 1 both using skin and thyroid transplantations.

As shown in Tables 4 and 5, Copolymer 1 inhibited graft rejection in all strain combinations as demonstrated by the prolongation of the skin graft survival as well as by the elevation in the thyroid iodine absorbance in the Copolymer 1-treated mice in comparison to the PBS-treated mice. Copolymer 1 significantly inhibited even the rejection of grafts from donors of different H-2 haplotypes (Tables 4 and 5), which usually induce a more potent rejection course than the rejection of H-2 matched transplants. These results indicate that Copolymer 1 is effective in suppressing immune rejection of grafts from various origins in different strain combinations, and thus may be effective in other species as well.

TABLE 4
Effect of Copolymer 1 treatment on skin graft rejection in various
haplotypes

| Haplotype | Treatment | N | MST* ± SD |
|---|---|---|---|
| B10D2→BALB | PBS | 26 | 16.1 ± 2.2 |
| (H-2^{d}) | | | |
| B10D2→BALB | Cop 1 | 34 | 20.6 ± 3.3 |
| (H-2^{d}) | | | |
| C57BL→C3HSW | PBS | 9 | 16.2 ± 1.0 |
| (H-2^{b}) | | | |
| C57BL→C3HSW | Cop 1 | 9 | 17.7 ± 2.1 |
| (H-2^{b}) | | | |
| B10PL→PL/J | PBS | 10 | 15.1 ± 3.5 |
| (H-2^{u}) | | | |
| B10PL→PL/J | Cop 1 | 9 | 17.6 ± 5.1 |
| (H-2^{u}) | | | |
| C57BL→C3HSW | PBS | 18 | 14.0 ± 1.8 |
| (H-2^{b} → H-2^{d}) | | | |
| C57BL→C3HSW | Cop 1 | 18 | 18.5 ± 3.3 |
| (H-2^{b} → H-2^{d}) | | | |

*Mean Survival Time.

TABLE 5
Effect of Cop 1 treatment on thyroid rejection in various haplotypes

| | | | Mean I¹²⁵ Absorption | | | | |
|---|---|---|---|---|---|---|---|
| | | | Trans planted | Untrans planted | | | |
| | Treatment | N | Kidney (cpm) | Kidney (cpm) | Δcpm | MFI* | P** |
| B10.D2 → BALB | PBS | 10 | 1193 | 421 | 772 | 1.0 | |
| (H-2^{d}) | | | | | | | |
| B10.D2 → BALB | Cop 1 | 10 | 2901 | 450 | 2451 | 3.2 | 0.0005 |
| (H-2^{d}) | | | | | | | |
| B10.D2 → BALB | PBS | 5 | 354 | 180 | 174 | 1.0 | |
| (H-2^{d}) | | | | | | | |
| B10.D2 → BALB | Cop 1 | 5 | 1035 | 137 | 898 | 5.2 | 0.0014 |
| (H-2^{d}) | | | | | | | |
| C57BL → C3H | PBS | 7 | 893 | 293 | 600 | 1.0 | |
| (H-2^{b}) | | | | | | | |
| C57BL → C3H | Cop 1 | 4 | 1643 | 281 | 1362 | 2.3 | 0.0023 |
| (H-2^{b}) | | | | | | | |
| B10PL → PL/J | PBS | 7 | 1201 | 518 | 683 | 1.0 | |
| (H-2^{d}) | | | | | | | |
| B10PL → PL/J | Cop 1 | 6 | 2009 | 332 | 1677 | 2.5 | 0.0016 |
| (H-2^{d}) | | | | | | | |
| C57BL → BALB | PBS | 8 | 4021 | 766 | 3255 | 1.0 | |
| (H-2^{b} → H-2^{d}) | | | | | | | |
| C57BL → BALB | Cop 1 | 10 | 10759 | 924 | 9835 | 3.0 | 0.002 |
| (H-2^{b} → H-2^{d}) | | | | | | | |

The mean function index (MFI) for Cop 1 treatment in each strain combination wsa calculated as follows:

$* MFI = \frac{mean Δcpm for the Cop 1 treated mice}{mean \underline{Δ} cpm for the PBS treated mice}$
**P values were obtained by analysis of variance (ANOVA).

### Example 3

### The Effect of Cop 1 Treatment on HVG in Comparison to Other Immunosuppressive Drugs

The effect of Cop 1 in comparison to the effect of two other immunosuppressive drugs that are currently used to prevent graft rejection in human transplantation, tacrolimus (FK506) and cyclosporin A (CyA), was tested in the two model systems.

BALB/c recipient mice were transplanted with skin grafts originated in B10.D2 donors and treated daily with: PBS ip from day −7, Cop 1 (ip+sc) from day −7, CyA ip from day −7, and FK 506 ip 7 injections from day −2 before transplantation. Grafts were inspected daily. Rejection was considered positive when no viable donor epidermis remained. Thyroid glands from B10.D2 donors were transplanted in the kidney's capsules of BALB/c mice. While CyA induced no significant beneficial effect in these systems, FK 506 significantly improved grafts survival/function in both the skin and the thyroid transplantation systems. Cop 1 also induced significant beneficial effect on graft survival/function similar to the effect of FK 506. While Cop 1 effect on skin graft survival was somewhat smaller than the effect of FK 506 (MST 20.6 for Cop 1 in comparison to 21.2 for FK 506 (Table 2), Cop 1 was as effective as FK 506, in preventing the functional deterioration of transplanted thyroid grafts (3.2 and 3.1 folds over the PBS control for Cop 1 and FK 506 respectively, Table 3 and FIG. 2).

### Example 4

### Effect of Cop 1 in Combination with FK-506 or CyA Treatment on Skin Draft Rejection in the C57BL→BALB/c Model

In order to test tested whether combined treatments of glatiramer acetate (GA) with various doses of CyA and FK 506 may extend skin graft survival in comparison to the effect of each drug alone. BALB/c recipient mice were transplanted with skin grafts originated in C57BL donors in day 0. GA (100 mg/kg, s.c.) was injected daily starting 2 weeks before transplantation. Tacrolimus (FK 506) and CyA in the indicated concentrations, were injected i.m. daily, starting 6 days before transplantation. Rejection was considered positive when no viable donor epidermis remained.

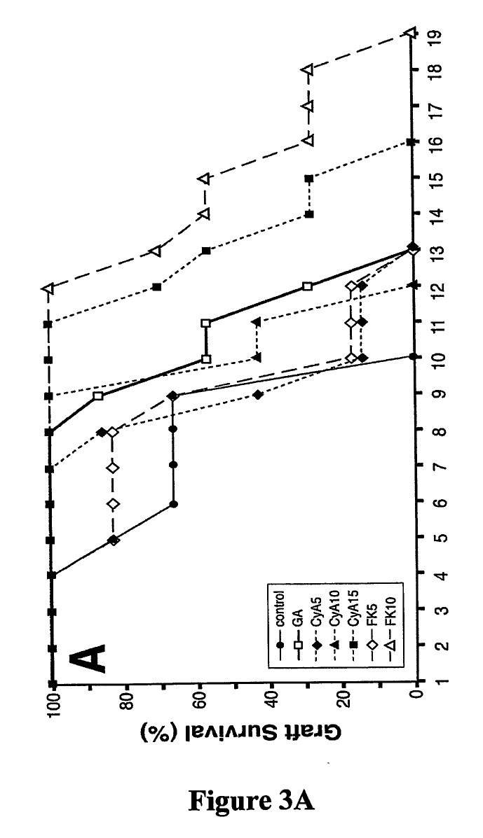

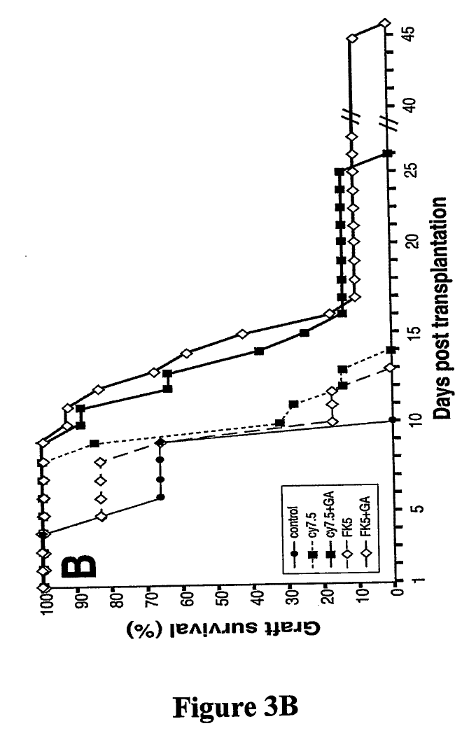

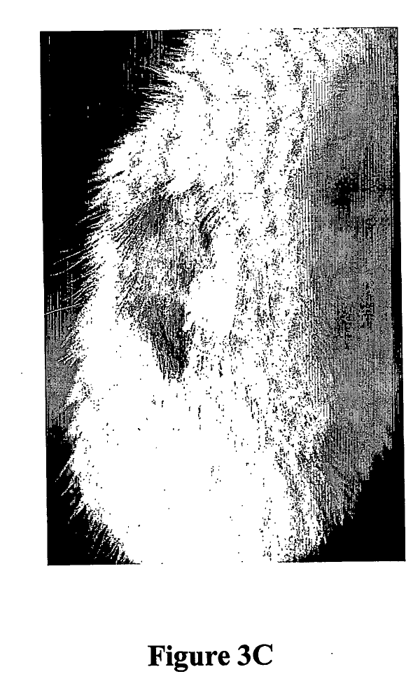

Table 6 indicates that combined treatment of GA with either CyA or tacrolimus prolonged significantly the graft survival in comparison to the effect of each drug alone. This was manifested in an additive effect—prolongation similar to the sum of the prolongation by each drug alone, when low doses of the immunosuppressive drugs were used (CyA 5 mg/kg and FK 1.25 mg/kg), and in synergistic effect—prolongation higher than the sum of the individual effect with higher immunosuppressant doses (CyA 7.5 mg/kg, FK 2.5 and 5 mg/kg). Thus, the combination effect (the ratio between the prolongation obtained with and without GA) of 3.0-5.4 and 2.1-3.0 fold was obtained for GA with CyA and GA with FK 506, respectively. The prolongation values obtained by the combined treatments were always higher than those obtained by a two fold higher doses of the same drug by itself. For example, combination of 7.5 mg/kg CyA with GA induced 38% prolongation in graft survival whereas a double dose −15 mg/kg CyA by itself induced only 27% prolongation. Similarly, combination of 5.0 mg/kg FK506 with GA induced 66% prolongation whereas treatment with 10 mg/kg FK 506 alone resulted in only 54% prolongation of skin graft survival. Interestingly, in each of these groups, treated by the combination of GA with either CyA 7.5 mg/kg or FK 506 5 mg/kg, one mouse survived for a prolonged period of time (25 days with GA and CyA and 45 days with GA and FK 506 respectively), even though treatment with the immuno-suppressive agent was discontinued 20 days after transplantation. In these cases, hair growth was observed in the skin grafts (FIG. 3C).

TABLE 6
The effect of GA in combination with immunosuppressive drugs on skin
graft rejection
A. Combination with Cyclosporin A

| Treatment | | | | | |
|---|---|---|---|---|---|
| CyA (mg/kg) | GA | N | MST* | ±SD | Prolongation (%) |
| none | − | 8 | 11.2 | ±1.2 | 0 |
| none | + | 7 | 12.3 | ±1.7 | 10 |
| 5.0 | − | 8 | 11.9 | ±1.2 | 6 |
| 5.0 | + | 8 | 13.2 | ±1.2** | 18 (×3.0) |
| 7.5 | − | 7 | 12.0 | ±2.0 | 7 |
| 7.5 | + | 9 | 15.5 | ±3.9** | 38^{#} (×5.4) |
| 15.0 | − | 7 | 14.2 | ±1.1** | 27 |

B. Combination with FK 506

| Treatment | | | | | |
|---|---|---|---|---|---|
| FK 506 (mg/kg) | GA | N | MST* | ±SD | Prolongation (%) |
| none | − | 16 | 10.4 | ±1.1 | 0 |
| none | + | 14 | 11.5 | ±0.4** | 11 |
| 1.25 | − | 7 | 11.2 | ±1.8 | 8 |
| 1.25 | + | 7 | 12.2 | ±1.5** | 17 (×2.1) |
| 2.5 | − | 8 | 11.8 | ±2.0 | 13 |
| 2.5 | + | 8 | 14.0 | ±2.5** | 35 (×2.7) |
| 5.0 | − | 16 | 12.7 | ±3.2** | 22 |
| 5.0 | + | 13 | 17.5 | ±2.7** | 68^{#} (×3.1) |
| 10 | − | 7 | 16.0 | ±3.0** | 54 |

*Mean Graft Survival Time (days).
**Indicates statistical significance p, 0.05.
^{#}In one mouse graft was rejected only after 45 days in combination with FK506, and after 38 days in combination with CyA. The numbers in parentheses indicate the combination effect - the MST ratio between two groups treated with the same concentration of FK 506 or CyA with and without the addition of GA.

FIG. 3 demonstrate the effect of GA, CyA and FK506 on skin graft rejection. Cop1 treatment induced significant prolongation of skin graft survival, with mean survival time (MST) 10.8 days, compared with 8.5 days obtained in the untreated control group (increase of 27%). This prolongation was longer than that obtained by CyA or FK 506 at 5 mg/kg—MST 9.7 and 9.5 days (prolongation of 14% and 12% respectively), and similar to that of 10 mg/kg CyA. GA was less effective than the highest doses of these drugs—CyA 15 mg/kg, or FK 506 10 mg/kg, with MST 16 and 13.8 days (62% and 88% prolongation), respectively. However, these higher doses of CyA as well as FK 506 had a toxic effect on the mice, inducing considerable weight loss and weakness (as depicted in FIG. 3A, and even mortality (an average of 20% of the mice). In contrast, mice injected daily with a much higher dose (100 mg/kg) of GA looked completely healthy (FIG. 3B).
A photograph of a particular mouse treated with combination of GA and FK 506 one month after transplantation is shown in FIG. 3C. The BALB/c Mouse was transplanted with skin graft from C57BL/6 donor and treated by the combined treatment of GA 100 mg/kg and FK506 5 mg/kg. The skin graft survived for 45 days even though treatment was discontinued 20 days after transplantation and even showed hair growth, indicating full engraftment. A similar effect was obtained in a mouse treated with combined treatment of GA 100 mg/kg and CyA 7.5 mg/kg which survived for 25 days.

### Example 5

### Effect of Cop 1 in Combination with FK-506 or CyA Treatment on the Function of Transplanted Thyroids in the C57BL→BALB/c Model

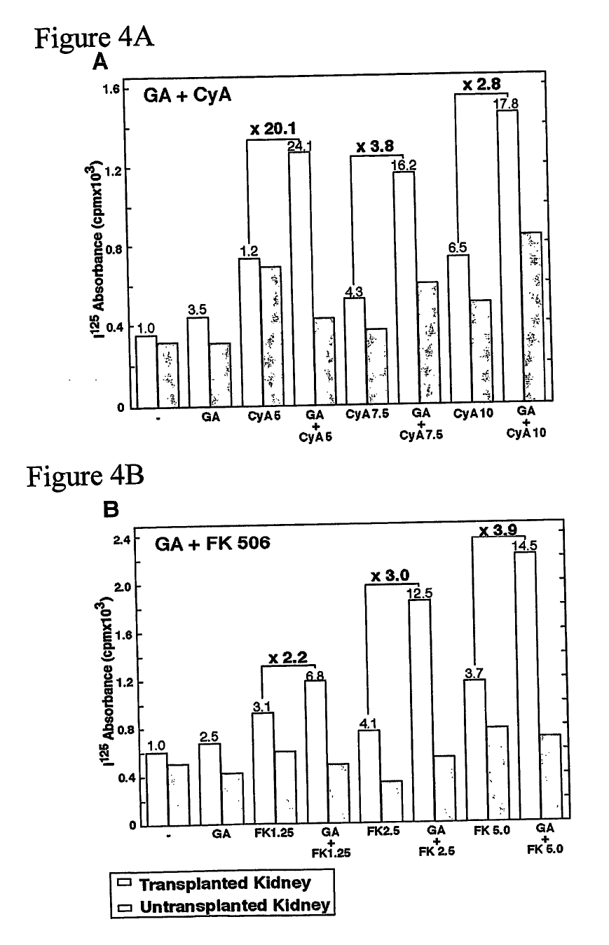

BALB/c recipient mice were transplanted in the kidney's capsules with thyroid glands from donor mice in day 0. Grafts were inspected daily. The function (iodine absorbance) of the transplanted thyroid tissue was examined 10 days after transplantation by measuring the iodine absorbance in the kidney. The results summarized in Table 7 below and in FIG. 4 revealed that the combination therapy of Cop 1 with either FK 506 or CyA significantly improved graft function, and was more effective than treatment with either Cop 1 or the immunosuppressive drug alone. Importantly, it is clear that the mean functional index (MFI) is significantly improved in animals receiving the combination therapy of Cop1 together with a known immunosuppressive drug.

In FIG. 4 the numbers present in the bars denote the Iodine uptake in the kidney with the transplant versus the untransplanted kidney. The numbers in bold between pairs of bars, denote the multiple increase in MFI achieved by the combination therapy versus the monotherapy. Notably, the combination of Cop 1 and Cyclosporin A at the lowest dose achieved over 20 fold improvement in MFI.

TABLE 7 (Part 1)
Combination therapy
Effect of Cop 1 and CyA treatment on the function of transplanted thyroids

| | | | Mean I¹²⁵ Absorbance | | | |
|---|---|---|---|---|---|---|
| | | | transplanted | untransplanted kidney | | |
| Treatment | | N | kidney (cpm) | (cpm) | Δcpm | MFI |
| PBS | | 5 | 353 ± 83 | 318 ± 74 | 35 | 1.0 |
| Cop 1 | 2.5 mg/mouse | 5 | 439 ± 31 | 317 ± 65 | 122 | 3.5 |
| CyA | 5 mg/kg | 4 | 740 ± 220 | 697 ± 496 | 43 | 1.2 |
| Cop 1 + CyA | 2.5 mg/mouse | 4 | 1279 ± 457 | 434 ± 153 | 845 | 24.1 |
| | 5 mg/kg | | | | | (×20.1) |
| CyA | 7.5 mg/kg | 5 | 529 ± 114 | 379 ± 65 | 150 | 4.3 |
| Cop 1 + CyA | 2.5 mg/mouse | 5 | 1173 ± 477 | 605 ± 358 | 568 | 16.2 |
| | 7.5 mg/kg | | | | | (×3.8) |
| CyA | 10 mg/kg | 4 | 743 ± 367 | 516 ± 176 | 227 | 6.5 |
| Cop 1 + CyA | 2.5 mg/mouse | 5 | 1476 ± 498 | 852 ± 235 | 624 | 17.8 |
| | 10 mg/kg | | | | | (×2.8) |

Thyroid glands from C57BL mice were transplanted in the kidney capsule of BALB/c mice. Nine days after transplantation, mice were injected with 1 μcI¹²⁵, and the radioactivity of each kidney was measured 20 hr. later.
MFI—denotes the mean function index, calculated as

MFI=mean Δcpm for the tested treatment/mean Δcpm for the PBS tested treatment

TABLE 7 (Part 2)
Combination Therapy
Effect of Cop 1 and FK506 treatment on the function of transplanted thyroids

| | | | Mean I¹²⁵ Absorbance | | | |
|---|---|---|---|---|---|---|
| | | | Transplanted | | | |
| | | | kidney | untransplanted kidney | | |
| Treatment | | N | (cpm) | (cpm) | Δcpm | MFI |
| PBS | | 4 | 605 ± 94 | 501 ± 161 | 104 | 1.0 |
| Cop 1 | 2.5 mg/mouse | 5 | 671 ± 24 | 424 ± 115 | 247 | 2.4 |
| FK506 | 1.25 mg/kg | 5 | 928 ± 165 | 601 ± 61 | 327 | 3.1 |
| Cop 1 + FK506 | 2.5 mg/mouse | 5 | 1193 ± 457 | 487 ± 133 | 706 | 6.8 |
| | 1.25 mg/kg | | | | | (×2.2) |
| FK506 | 2.5 mg/kg | 5 | 772 ± 519 | 341 ± 108 | 431 | 4.1 |
| Cop 1 + FK506 | 2.5 mg/mouse | 5 | 1856 ± 650 | 551 ± 75 | 1305 | 12.5 |
| | 2.5 mg/kg | | | | | (×3.0) |
| FK506 | 5 mg/kg | 5 | 1167 ± 636 | 784 ± 394 | 383 | 3.7 |
| Cop 1 + FK506 | 2.5 mg/mouse | 5 | 2233 ± 1729 | 720 ± 250 | 1513 | 14.5 |
| | 5 mg/kg | | | | | (×3.9) |

MFI denotes the mean function index calculated as

$$MFI = \frac{mean Δ cpm for the tested treatment}{mean Δ cpm for the PBS treatment}$$

### Example 6

### Effect of Cop 1 in Combination with CyA Treatment on Heart Rejection in Rats

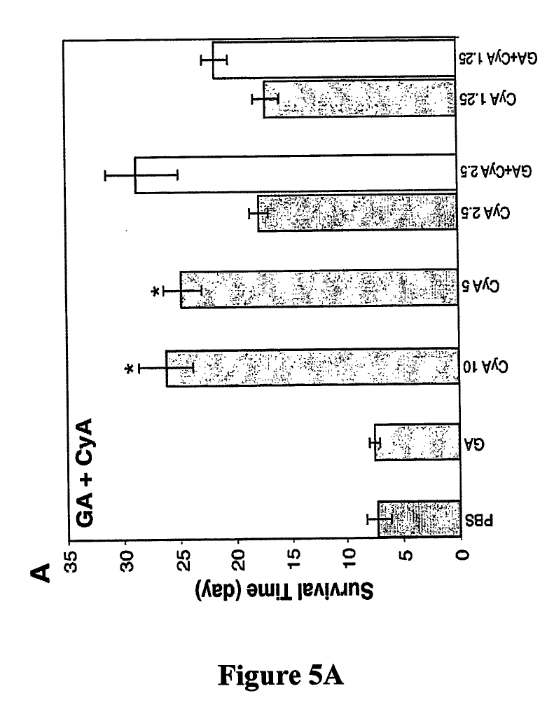

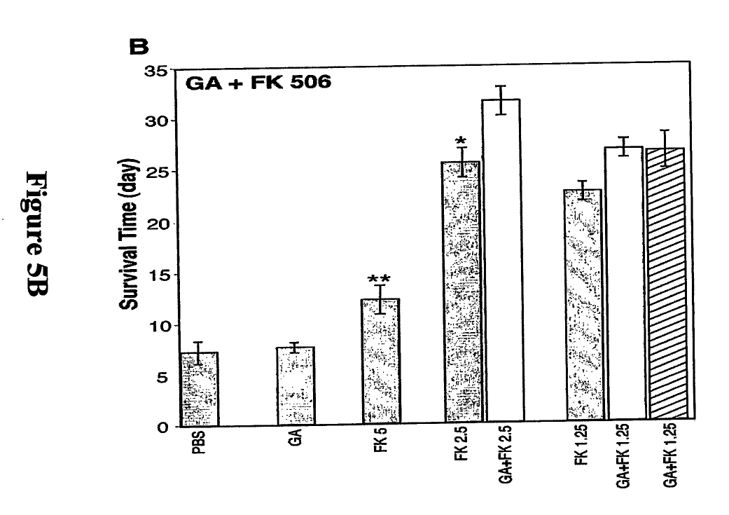

Lewis rats were transplanted with an accessory heart from the allogeneic disparate BN donor rats. Recipient rats were treated daily with either GA 100 mg/kg starting 2 weeks before transplantation; CyA 1.25, 2.5, 5 or 10 mg/kg starting on the day of transplantation; or FK506 1.25, 2.5 or 5 mg/kg starting 6 days before transplantation (FIG. 5, shaded bars). Combination of pretreatment with GA and the respective doses of the immunosuppressants (open bars); Treatment with GA administered from the day of transplantation without pretreatment (striped bar). Cardiac allograft survival was inspected by daily monitoring palpation of the grafts. Grafts were considered rejected when no heart palpitations could be observed.

The mean survival time of the transplanted hearts in recipient, rats treated with GA, CyA, FK 506 as well as by their combinations is demonstrated in Table 8 and FIG. 5. As demonstrated, in this system, GA treatment by itself did not prolong graft survival, whereas the different concentrations of CyA and FK 506 induced significant prolongation, in a dose dependent manner. Yet, the doses in which considerable suppressive effects were obtained (5 or 10 mg/kg CyA and 2.5 or 5 mg/kg FK 506) had also toxic consequences and rats receiving 5 mg/kg FK 506 even died after 6-8 daily injections. The addition of GA to either CyA or FK 506 treatment significantly extended heart survival beyond the effect of each drug alone. Thus, an average of 28.7 days survival was achieved by the combination of GA with 2.5 mg/kg CyA (prolongation of 21.5 days in comparison to untreated control), whereas, the same dose of CyA alone led to survival of only 17.8 days (prolongation of 10.6 days) pointing at 2 fold prolongation effect by the combined treatment (FIG. 5A and Table 8). Moreover, the 28.7 days survival with combination of GA and 2.5 mg/kg CyA was longer than the 2,6.2 days obtained with four fold higher dose of CyA alone, which was considerably toxic. Likewise, combined treatment of GA with FK 506, at 1.25 and 2.5 mg/kg, resulted in 26.7 and 31.5 days survival, respectively in comparison to 22.5 and 25.5 days obtained by the same doses of FK 506 alone (FIG. 5B and Table 8). As demonstrated in FIG. 5, similar results were obtained when the combination of Cop1 and CyA (2.5 mg/kg) was administered compared to administration CyA alone in a much higher dose (i.e. 10 mg/kg). Same pattern was obtained with combination of Cop1 and FK 506.

Most importantly, the combination of GA with FK 506 was similarly effective when GA administration started together with the FK506 on the day of transplantation without pretreatment. For FK 506, as in the case of CyA, the combination with GA resulted in longer survival than those obtained by higher, toxic, doses of the drug alone.

TABLE 8
The effect of Cop1 in combination with immunosuppressive drugs on heart
rejection
A. Combination with Cyclosporin A

Treatment

| CyA | | | | Prolongation |
|---|---|---|---|---|
| (mg/kg) | GA | N | MST* ± SD | (days) |
| none | − | 15 | 7.2 ± 1.1 | 0 |
| none | + | 5 | 7.6 ± 0.5 | 0.4 |
| 1.25 | − | 6 | 17.1 ± 1.2 | 9.9 |
| 1.25 | + | 5 | 21.6 ± 1.1 | 14.4 (×1.5) |
| 2.5 | − | 5 | 17.8 ± 0.8 | 10.6 |
| 2.5 | + | 12 | 28.7 ± 3.7 | 21.5 (×2.0) |
| 5.0 | − | 7 | 24.7 ± 1.7 (toxic) | 17.5 |
| 10 | − | 8 | 26.2 ± 2.4 (toxic) | 19.2 |

B. Combination with FK 506

| Treatment | | | | |
|---|---|---|---|---|
| FK 506 | | | | Prolongation |
| (mg/kg) | GA | N | MST* ± SD | (days) |
| none | − | 15 | 7.2 ± 1.1 | 0 |
| none | + | 5 | 7.6 ± 0.5 | 0.4 |
| 1.25 | − | 6 | 22.5 ± 0.9 | 15.3 |
| 1.25 | + | 6 | 26.7 ± 0.9 | 19.5 (×1.3) |
| 1.25 | + | 6 | 26.6 ± 1.7 | 19.4 (×1.3) |
| | (no pretreatment) | | | |
| 2.5 | − | 6 | 25.5 ± 1.4 (toxic) | 18.3 |
| 2.5 | + | 6 | 31.5 ± 1.4 | 24.3 (×1.3) |
| 5.0 | − | 6 | toxic all rats died | — |

*Mean Graft Survival Time (days). The numbers in parenthesis indicate the combination effect - the MST ratio between two groups treated with the same concentration of FK 506 or CyA with and without the addition of GA.

It will be appreciated by a person skilled in the art that the present invention is not limited by what has been particularly shown and described hereinabove. Rather, the scope of the invention is defined by the claims that follow.

### REFERENCES

Aharoni et al., “T suppressor hybridomas and interleukin-2-dependent lines induced by copolymer 1 or by spinal cord homogenate down-regulate experimental allergic encephalomyelitis”, Eur. J. Immunol. 23:17-25 (1993).
Aharoni et al., “Studies on the mechanism and specificity of the effect of the synthetic random copolymer GLAT on graft-versus-host disease”, Immunology Letters, 58(2):79-87 (1997).
Aharoni R, Teitelbaum D, Sela M, Amon R. Copolymer 1 induces T cells of the T helper type 2 that crossreact with myelin basic protein and suppress experimental autoimmune encephalomyelitis. Proc. Natl. Acad. Sci. USA. 94: 10821 (1997).
Aharoni R, Teitelbaum D, Sela M, Amon R. Copolymer 1 inhibits manifestation of graft rejection. Transplantation 72:598 (2001).
Bornstein et al., “Clinical trials of Cop 1 in multiple sclerosis” Handbook of Multiple Sclerosis, ed. Cook S. D. Marcel Dekker, Inc., p. 469 (1990).
Fridkis-Hareli et al., “Direct binding of myelin basic protein and synthetic copolymer 1 to class II major histocompatibility complex molecules on living antigen-presenting cells—specificity and promiscuity” Proc. Natl. Acad. Sci. USA. 91: 4872-76 (1994).
Isakov et al., “Differential immunogenic expression of an H-2-linked histocompatibility antigen on different tissues. Differences in survival between heart, thyroid, and skin allografts”, Transplantation 28(1):31-5 (1979).
Ishioka et al., “Failure to demonstrate long-lived MHC saturation both in vitro and in vivo. Implications for therapeutic potential of MHC-blocking peptides”, J. Immunol., 152(9):4310-4319 (1994).
Jacob et al., “DNA polymorphism in cytokine genes based on length variations in simple-sequence tandem repeats” Immunogenetics, 38: 251 (1993).
Johnson et al., “Cop 1 positive results—a phase III trial in relapsing remitting” MS. 11 Annual Meeting A.N.A. (1994).
Johnson K P, Brooks B R, Cohen J A, et al. Extended use of Glatiramer Acetate (Copaxone) is well tolerated and maintains its clinical effect on Multiple Sclerosis relapse rate and degree of disability. Neurology 50: 701 (1998).
Johnson et al., “Copolymer 1 reduces relapse rate and improves disability in relapsing-remitting multiple sclerosis: results of a phase III multicenter, double-blind placebo-controlled trial. The Copolymer 1 Multiple Sclerosis Study Group.” Neurology 1:65 (1995).
Morris P J, Wood K J, Dallman M J. Antigen-induced tolerance to organ allografts. Annals of the New York Academy of Science 636: 295 (1991).
Schlegel et al., “Prevention of Graft-Versus-Host Disease by Peptides Binding to Class II Major Histocompatibility Complex Molecules” Blood 84: 2802-1.0 (1994).
Schlegel et al., “A synthetic random basic copolymer with promiscuous binding to class II major histocompatibility complex molecules inhibits T-cell proliferative responses to major and minor histocompatibility antigens in vitro and confers the capacity to prevent murine graft-versus-host disease in vivo”, Proc. Natl. Acad. Sci. (USA), 93:5061-6 (1996).
Sela et al., Bull. Inst. Pasteur (Paris) 88: 303-314 (1990).
Sykes et al, “Immunobiology of transplantation”, FASEB J. 10(7):721-30 (1996).
Teitelbaum et al., “Suppression of experimental allergic encephalomyelitis by a synthetic polypeptide” Eur. J. Immunol. 1:242-48 (1971).
Teitelbaum et al., “Suppression by several synthetic polypeptides of experimental allergic encephalomyelitis induced in guinea pigs and rabbits with bovine and human basic encephalitogen” Eur J. Immunol. 3:272 (1973).
Teitelbaum et al., “Suppression of experimental allergic encephalomyelitis in rhesus monkeys by a synthetic basic copolymer” Clin. Immunol. Immunopathol. 3:256 (1974a).
Teitelbaum et al., “Suppression of experimental allergic encephalomyelitis in baboons by Cop 1” Israel J. Med. Sci. 13:1038 (1974b).
Teitelbaum D, Aharoni R, Fridkis-Hareli M, Amon R, Sela M. Development of copolymer 1 (Copaxone) as a specific drug against multiple sclerosis. In: Shoenfeld Y, ed. The Decade in Autoimmunity. Elsevier, 183 (1998).
Teitelbaum et al., “Specific inhibition of the T-cell response to myelin basic protein by the synthetic copolymer Cop 1”, Proc. Natl. Acad. Sci USA 85:9724-28 (1988).
Webb et al., “Molecular requirements involved in suppression of EAE by synthetic basic copolymers of amino acids”, Immunochemistry 13:333-337 (1976).
Webb et al., “Extrathymic tolerance of mature T cells: clonal elimination as a consequence of immunity”, Cell, 63:1249-1256 (1990).

1. A method of treating or preventing graft rejection in a subject in need thereof, comprising administering a therapeutically effective amount of at least one copolymer 1 or copolymer 1-related heteropolymer in combination with at least one immunosuppressive drug, said copolymer 1 or copolymer 1-related heteropolymer comprises at least three amino acids each one selected from at least three of the following groups:
(a) lysine and arginine;
(b) glutamic acid and aspartic acid;
(c) alanine, glycine and valine;
(d) tyrosine, tryptophan and phenylalanine.

2. The method of claim 1, wherein said copolymer 1 or copolymer 1-related heteropolymer is selected from the group consisting of a random heteropolymer, an ordered heteropolymer, or an ordered peptide.

3. The method of claim 2, wherein said ordered peptide is selected from SEQ ID NOS: 1-32.

4. The method of claim 1, wherein said immunosuppressive drug is selected from the group consisting of antiproliferative drugs, inhibitors of lymphocyte activation, steroids, purine antimetabolites, antibodies and immunomodulators.

5. The method of claim 4, wherein said immunosuppressive drug is cyclosportin A, FK 506, ISA247, FK 778, rapamycin, everolimus or FTY720.

6. The method of claim 1, wherein said graft rejection is associated with transplantation of cells, tissue or organs, selected from hematopoietic cells, stem cells, heart, lung, kidney, liver or skin.

7. The method of claim 1, wherein said therapeutically effective amounts of copolymer 1 or copolymer 1-related heteropolymer and said at least one immunosuppressive drug are administered together or sequentially.

8. The method of claim 2, wherein said copolymer contains four different amino acids each one selected from the groups (a) to (d).

9. The method of claim 8, wherein said heteropolymer comprises in combination alanine, glutamic acid, lysine, and tyrosine, of net overall positive electrical charge and of a molecular weight of about 2,000 to about 40,000 daltons.

10. The method of claim 9, wherein said heteropolymer has a molecular weight of about 2,000 to about 13,000 daltons.

11. The method of claim 2, wherein said heteropolymer contains three different amino acids each one selected from the groups (a) to (d), herein referred to as terpolymer.

12. The method of claim 11, wherein said terpolymer consists essentially of the amino acids tyrosine, alanine and lysine.

13. The method of claim 12, wherein said terpolymer consists essentially of tyrosine, alanine and lysine, in the molar ratio of from about 0.005 to about 0.25 tyrosine, from about 0.3 to about 0.6 alanine, and from about 0.01 to about 0.5 lysine, herein designated YAK.

14. The method of claim 11, wherein said terpolymer consists essentially of the amino acids glutamic acid, tyrosine and lysine.

15. The method of claim 14, wherein said terpolymer consists essentially of the amino acids glutamic acid, tyrosine, and lysine in the molar ratio of from about 0.005 to about 0.300 glutamic acid, from about 0.005 to about 0.250 tyrosine, and from about 0.3 to about 0.7 lysine, herein designated YEK.

16. The method of claim 11, wherein said terpolymer consists essentially of the amino acids tyrosine, glutamic acid, and alanine.

17. The method of claim 16, wherein said terpolymer consists essentially of the amino acids tyrosine, glutamic acid, and alanine in the molar ratio of from about 0.005 to about .0.25 tyrosine, from about 0.005 to about 0.3 glutamic acid, and from about 0.005 to about 0.8 alanine, herein designated YEA.

18. The method according to claim 2, wherein the amino acids comprising the heteropolymer are all L-, all D- or a mixture of L- and D-amino acids.

19. A pharmaceutical composition for use in preventing and treating graft rejection, comprising a therapeutically effective amount of at least one synthetic peptide copolymer selected from glatiramer acetate and a copolymer 1-related heteropolymer according to claim 1 in combination with at least one immunosuppressive drug and a pharmaceutically acceptable carrier.

20. The pharmaceutical composition of claim 19 in unit dosage form suitable for oral administration.

21. The pharmaceutical composition of claim 19 in unit dosage form suitable for parenteral injection.

22. A method of treating or preventing graft rejection in a subject in need thereof, comprising administering a therapeutically effective amount of at least one ordered peptide or ordered copolymer 1-related heteropolymer, said ordered peptide or ordered copolymer 1-related heteropolymer comprising at least three amino acids each one selected from at least three of the following groups:
(a) lysine and arginine;
(b) glutamic acid and aspartic acid;
(c) alanine, glycine and valine;
(d) tyrosine, tryptophan and phenylalanine.

23.-35. (canceled)

36. The method of claim 22, wherein said ordered copolymer is an oligomer of at least one ordered peptide selected from SEQ ID NOS: 1-32.

37.-38. (canceled)


SEQUENCE LISTING (32 sequences)

SEQ ID NO: 1 - protein, 15 aa, Artificial sequence
  1..15: synthetic peptide
AAAYAAAAAAKAAAA

SEQ ID NO: 2 - protein, 15 aa, Artificial sequence
  synthetic peptide
AEKYAAAAAAKAAAA

SEQ ID NO: 3 - protein, 15 aa, Artificial sequence
  synthetic peptide
AKEYAAAAAAKAAAA

SEQ ID NO: 4 - protein, 15 aa, Artificial sequence
  synthetic peptide
AKKYAAAAAAKAAAA

SEQ ID NO: 5 - protein, 15 aa, Artificial sequence
  synthetic peptide
AEAYAAAAAAKAAAA

SEQ ID NO: 6 - protein, 15 aa, Artificial sequence
  synthetic peptide
KEAYAAAAAAKAAAA

SEQ ID NO: 7 - protein, 15 aa, Artificial sequence
  synthetic peptide
AEEYAAAAAAKAAAA

SEQ ID NO: 8 - protein, 15 aa, Artificial sequence
  synthetic peptide
AAEYAAAAAAKAAAA

SEQ ID NO: 9 - protein, 15 aa, Artificial sequence
  synthetic peptide
EKAYAAAAAAKAAAA

SEQ ID NO: 10 - protein, 15 aa, Artificial sequence
  synthetic peptide
AAKYEAAAAAKAAAA

SEQ ID NO: 11 - protein, 15 aa, Artificial sequence
  synthetic peptide
AAKYAEAAAAKAAAA

SEQ ID NO: 12 - protein, 15 aa, Artificial sequence
  synthetic peptide
EAAYAAAAAAKAAAA

SEQ ID NO: 13 - protein, 15 aa, Artificial sequence
  synthetic peptide
EKKYAAAAAAKAAAA

SEQ ID NO: 14 - protein, 15 aa, Artificial sequence
  synthetic peptide
EAKYAAAAAAKAAAA

SEQ ID NO: 15 - protein, 15 aa, Artificial sequence
  synthetic peptide
AEKYAAAAAAAAAAA

SEQ ID NO: 16 - protein, 15 aa, Artificial sequence
  synthetic peptide
AKEYAAAAAAAAAAA

SEQ ID NO: 17 - protein, 15 aa, Artificial sequence
  synthetic peptide
AKKYEAAAAAAAAAA

SEQ ID NO: 18 - protein, 15 aa, Artificial sequence
  synthetic peptide
AKKYAEAAAAAAAAA

SEQ ID NO: 19 - protein, 15 aa, Artificial sequence
  synthetic peptide
AEAYKAAAAAAAAAA

SEQ ID NO: 20 - protein, 15 aa, Artificial sequence
  synthetic peptide
KEAYAAAAAAAAAAA

SEQ ID NO: 21 - protein, 15 aa, Artificial sequence
  synthetic peptide
AEEYKAAAAAAAAAA

SEQ ID NO: 22 - protein, 15 aa, Artificial sequence
  synthetic peptide
AAEYKAAAAAAAAAA

SEQ ID NO: 23 - protein, 15 aa, Artificial sequence
  synthetic peptide
EKAYAAAAAAAAAAA

SEQ ID NO: 24 - protein, 15 aa, Artificial sequence
  synthetic peptide
AAKYEAAAAAAAAAA

SEQ ID NO: 25 - protein, 15 aa, Artificial sequence
  synthetic peptide
AAKYAEAAAAAAAAA

SEQ ID NO: 26 - protein, 15 aa, Artificial sequence
  synthetic peptide
EKKYAAAAAAAAAAA

SEQ ID NO: 27 - protein, 15 aa, Artificial sequence
  synthetic peptide
EAKYAAAAAAAAAAA

SEQ ID NO: 28 - protein, 15 aa, Artificial sequence
  synthetic sequence
AEYAKAAAAAAAAAA

SEQ ID NO: 29 - protein, 15 aa, Artificial sequence
  synthetic peptide
AEKAYAAAAAAAAAA

SEQ ID NO: 30 - protein, 15 aa, Artificial sequence
  synthetic peptide
EKYAAAAAAAAAAAA

SEQ ID NO: 31 - protein, 15 aa, Artificial sequence
  synthetic peptide
AYKAEAAAAAAAAAA

SEQ ID NO: 32 - protein, 15 aa, Artificial sequence
  synthetic peptide
AKYAEAAAAAAAAAA